(19) United States
(12) Patent Application Publication
(10) Pub. No.: US 2021/0043273 A1
(43) Pub. Date: Feb. 11, 2021

(54) METHODS, SYSTEMS, AND MEDIA FOR PREDICTING FUNCTIONS OF MOLECULAR SEQUENCES

(71) Applicants: Alexander T Taguchi, Cambridge, MA (US); Arizona Board of Regents on behalf of Arizona State University, Scottsdale, AZ (US)

(72) Inventors: Neal W Woodbury, Tempe, AZ (US); Alexander T Taguchi, Cambridge, MA (US)

(21) Appl. No.: 16/967,070

(22) PCT Filed: Feb. 4, 2019
(86) PCT No.: PCT/US19/16540

§ 371 Date: Aug. 3, 2020

Related U.S. Application Data
(60) Provisional application No. 62/625,867, filed on Feb. 2, 2018.
(60) Provisional application No. 62/650,342, filed on Mar. 30, 2018.

(51) Int. Cl. G16B 20/00; G16B 15/30; G16B 40/00; G16B 30/00

(52) U.S. Cl. CPC G16B 20/00; G16B 30/00; G16B 40/00; G16B 15/30

(57) ABSTRACT

Methods and systems for predicting functions of molecular sequences, comprising: generating an array that represents a sequence of molecules; determining a projection of the sequence of molecules, wherein the determining comprises multiplying a representation of the array that represents the sequence of the molecules by a first hidden layer matrix that represents a number of possible sequence dependent functions, wherein the first hidden layer matrix is determined during training of a neural network; and determining a function of the sequence of molecules by applying a plurality of weights to a representation of the projection of the sequence of molecules, wherein the plurality of weights is determined during the training of the neural network.

### CROSS REFERENCE TO RELATED APPLICATIONS

This application claims the benefit of U.S. Provisional Patent Application No. 62/625,867, filed Feb. 2, 2018, and U.S. Provisional Patent Application No. 62/650,342, filed Mar. 30, 2018, each of which is hereby incorporated by reference herein in its entirety.

### STATEMENT REGARDING GOVERNMENT FUNDED RESEARCH

This invention was made with government support under Grant No. HSHQDC-15-C-B0008 awarded by the Department of Homeland Security. The government has certain rights in the invention.

### TECHNICAL FIELD

The disclosed subject matter relates to methods, systems, and media for predicting functions of molecular sequences.

### BACKGROUND

Most approaches to relating the covalent structure of molecules in libraries to their function rely on the concept that the molecules can be described as a series of component pieces and those component pieces act more or less independently to give rise to function. A common example in the application of nucleic acid and peptide libraries is the derivation of a consensus motif, a description of a sequence of nucleotides or amino acids that assigns a position dependent functional significance to each. However, many of the interactions in biology cannot be described by such simple models and methods and higher order interactions between multiple components of a library molecule must be considered, both adjacent in the structure and distributed within the structure, with the ligand or functional activity in question. These higher order interactions are information rich processes, and thus to identify them requires the analysis of a large number of examples of interactions between the functional activity and many different library molecules.

The difficulty in designing models that do this accurately is that the models need to include high order interactions while at the same time not creating so many free parameters in the system so as to cause the problem to be under-determined.

Accordingly, it is desirable to provide new methods, systems, and media for predicting functions of molecular sequences.

### SUMMARY

Methods, systems, and media for predicting functions of molecular sequences are provided. In some embodiments, methods for predicting functions of molecular sequences are provided, the methods comprising: generating an array that represents a sequence of molecules; determining a projection of the sequence of molecules, wherein the determining comprises multiplying a representation of the array that represents the sequence of the molecules by a first hidden layer matrix that represents a number of possible sequence dependent functions, wherein the first hidden layer matrix is determined during training of a neural network; and determining a function of the sequence of molecules by applying a plurality of weights to a representation of the projection of the sequence of molecules, wherein the plurality of weights is determined during the training of the neural network.

In some embodiments, systems for predicting functions of molecular sequences are provided, the systems comprising: a memory; and a hardware processor coupled to the memory and configured to: generate an array that represents a sequence of molecules; determine a projection of the sequence of molecules, wherein the determining comprises multiplying a representation of the array that represents the sequence of the molecules by a first hidden layer matrix that represents a number of possible sequence dependent functions, wherein the first hidden layer matrix is determined during training of a neural network; and determine a function of the sequence of molecules by applying a plurality of weights to a representation of the projection of the sequence of molecules, wherein the plurality of weights is determined during the training of the neural network.

### BRIEF DESCRIPTION OF THE DRAWINGS

Various objects, features, and advantages of the disclosed subject matter can be more fully appreciated with reference to the following detailed description of the disclosed subject matter when considered in connection with the following drawings, in which like reference numerals identify like elements.

FIG. 1 shows an example of a process for predicting functions of molecular sequences using a single-hidden-layer neural network in accordance with some embodiments of the disclosed subject matter.

FIG. 2 shows an example of a process for predicting functions of molecular sequences using a two-hidden-layer neural network in accordance with some embodiments of the disclosed subject matter.

FIG. 3 shows an example of a technique for rectifying a function in accordance with some embodiments of the disclosed subject matter.

FIG. 4 shows an example of a process for creating an orthogonal eigensequence matrix in accordance with some embodiments of the disclosed subject matter.

FIG. 5 shows an example of predicting a binding value for a sequence of peptides in accordance with some embodiments of the disclosed subject matter.

FIG. 6 shows an example of extrapolating a binding value for a sequence of peptides in accordance with some embodiments of the disclosed subject matter.

FIG. 7 shows an example of predicting a cognate epitope of a monoclonal antibody in accordance with some embodiments of the disclosed subject matter

FIG. 8 shows a schematic diagram of an example of a system for predicting functions of molecular sequences in accordance with some embodiments of the disclosed subject matter.

FIG. 9 shows an example of hardware that can be used in a server and/or a user device in accordance with some embodiments of the disclosed subject matter.

FIG. 10 shows an example of fitting results using the binding pattern of the extracellular portion of the protein PD1 (programmed death 1) to a peptide array with ˜125,000 unique peptide sequences in accordance with some embodiments of the disclosed subject matter.

FIG. 11 shows an example of fitting results using the binding pattern of the extracellular portion of the protein PDL1 (programmed death ligand 1) to a peptide array with ˜125,000 unique peptide sequences in accordance with some embodiments of the disclosed subject matter.

FIG. 12 shows an example of fitting results using the binding pattern of the protein TNFα (tumor necrosis factor alpha) to a peptide array with ˜125,000 unique peptide sequences in accordance with some embodiments of the disclosed subject matter.

FIG. 13 shows an example of fitting results using the binding pattern of the extracellular portion of the protein TNFR2 (TNFα receptor 2) to a peptide array with ˜125,000 unique peptide sequences in accordance with some embodiments of the disclosed subject matter.

FIGS. 14A-14C show examples of scatter plots of predicted values versus measured values for 10% of the peptide/binding value pairs that were not involved in training a network in accordance with some embodiments of the disclosed subject matter.

FIGS. 15A-15C show examples of similarity matrices between amino acids used to construct peptides on the arrays in FIGS. 14A-14C, respectively, in accordance with some embodiments of the disclosed subject matter.

FIG. 16A shows an example of the Pearson Correlation between predicted and observed binding data as a function of the size of the training set in the example discussed in FIGS. 14A-14C and 15A-15C in accordance with some embodiments of the disclosed subject matter.

FIG. 16B shows an example of the Pearson Correlation between predicted and observed as a function of the number of descriptors used by the neural network to describe each amino acid in the example discussed in FIGS. 14A-14C and 15A-15C in accordance with some embodiments of the disclosed subject matter.

FIG. 17 shows an example of predicted versus measured values for diaphorase training only on weak binding peptides and predicting the strong binders in accordance with some embodiments of the disclosed subject matter.

FIG. 18 shows an example of a prediction of the ratio between diaphorase binding and binding to total serum protein depleted of IgG in accordance with some embodiments of the disclosed subject matter.

FIG. 19 shows an example of a prediction of the z-score between diaphorase with and without FAD bound in accordance with some embodiments of the disclosed subject matter.

FIG. 20 shows an example of a process that can generate the results shown in FIGS. 10-13 in accordance with some embodiments.

### DETAILED DESCRIPTION

In accordance with various embodiments, mechanisms (which can include methods, systems, and media) for predicting functions of molecular sequences are provided.

In some embodiments, the mechanisms described herein can be used to take data associated with chemical structure information, such as a sequence of monomers in a polymer, and create a neural network that allows the prediction of the function of sequences not in the original library.

In some embodiments, the mechanisms described herein can use a single set of specific molecular components and a single connecting reactionary chemistry for a large number of potential applications (examples given below), and a single manufacturing process and instrument can be used to create the molecules for any of these applications.

For example, in some embodiments, a molecular library can be created (as described in more detail below), which can include information for any suitable number of molecules (e.g., thousands of molecules, millions of molecules, billions of molecules, trillions of molecules, and/or any other suitable number), and the functional attributes of some or all of the molecules in the molecular library can be measured for any suitable function (e.g., binding, and/or any other suitable function as described below in more detail). Therefore, the large number of molecules described in the molecular library can provide diversity to create a quantitative relationship between structure and function that can then be used to design an optimized arrangement of the same molecular components used to create the library such that the new arrangement gives rise to enhanced function. For example, in some embodiments, the library can provide information between a molecular structure and a desired function, which can be used to design a new molecule that is not included in the library. Additionally, in instances where the molecular components and the number of components linked together by one or a small number of chemical bonds covers a sufficiently diverse functional space, the library can be used for any suitable purpose, as described below in more detail. Additionally, in instances where the molecular components are linked together by one common type of chemical bond (e.g., a peptide bond linking amino acids, and/or any other suitable type of chemical bond), any functional molecule designed in this way can be made with the same solid-state synthetic approach and using the same molecular components, thereby rendering manufacturing common to all compounds designed in this way. Therefore, the mechanisms described herein can be used to facilitate the equitable distribution of drugs globally and to play a positive role in personalized medicine applications, where an increasingly large number of different drugs, or combinations of drugs, may need to be generated for person-specific applications.

As a more particular example, in some embodiments, a molecular recognition profile of a target of a drug can be measured without the drug (e.g., a binding of the target to each of the molecules in a molecular library). Additionally, the molecular recognition profile of the target with the drug can be measured. The mechanisms described herein can then be used to design molecules that bind in the same place as the drug to identify molecules that can potentially replace the drug. In some such embodiments, each identified drug can then be synthesized using a single process and/or using a single manufacturing line changing only the sequence of a set of molecular components.

In some embodiments, the mechanisms described herein can be used for any suitable applications. For example, in some embodiments, the mechanisms can be used to: design new molecular libraries with specific functions; screen complex molecular systems of known structure for functional prediction; predict potential lead compounds with desirable functions; develop and implement diagnostic methods; develop therapeutics and vaccines; and/or for any other suitable applications. More particular examples of applications of the techniques described herein can include: the discovery/design of lead compounds to be used in the development of therapeutics; the discovery/design of potential targets of therapeutic treatment; the characterization of specific antibodies, such as monoclonal antibodies used as therapeutics, to determine what peptide and protein sequences they are expected to bind; the discovery/design of protein antigens that could be used in the development of vaccines; the discovery/design of ligands appropriate for developing specific binding complexes; the discovery/design of ligands that can be used to modify enzyme reactions; the discovery/design of ligands that can be used in the construction of artificial antibodies; the discovery/design of ligands that specifically interfere with binding between two targets; the discovery/design of binding partners (natural or man-made) to a particular target; the discovery/design of drugs such as antimicrobial drugs and/or any other suitable type of drugs; the design of peptide arrays that bind to specific antibodies or to serum with specific properties (e.g., the presence of antibodies expressed during a disease state); the enhancement and amplification of the diagnostic and prognostic signals provided by peptide arrays for use in analyzing the profile of antibodies in the blood produced in response to a disease, condition, or treatment; the discovery/design of protein antigens or polypeptide sequences that are responsible for the response to a disease, condition, or treatment (e.g., discovery of antigens for a vaccine); the discovery/design of protein antigens or polypeptide sequences that are responsible for adverse reactions resulting from a disease, condition, or treatment (e.g., autoimmune reactions); the design of coatings; the design of catalytic modifiers; the design of molecules for neutralization of toxic or unwanted chemical species; the design of adjuvants; the design of media for chromatography or purification; and/or for any other suitable applications.

As a more particular example, in some embodiments, pharmokinetics and solubility can be measured for a representative sample of molecular component combinations and used to predict pharmokinetic and solubility properties for all possible combinations. As a specific example, in the field of drug development, all drugs derived from this approach can have the same manufacturing system and many aspects of the drugs' action (e.g., toxicity, pharmokinetics, solubility, and/or any other suitable properties) can be accurately predicted based on previously known data about a molecular library (rather than about the specific application of the drug). Therefore, a drug specific to a particular application can be designed from simple, molecular-array-based measurements.

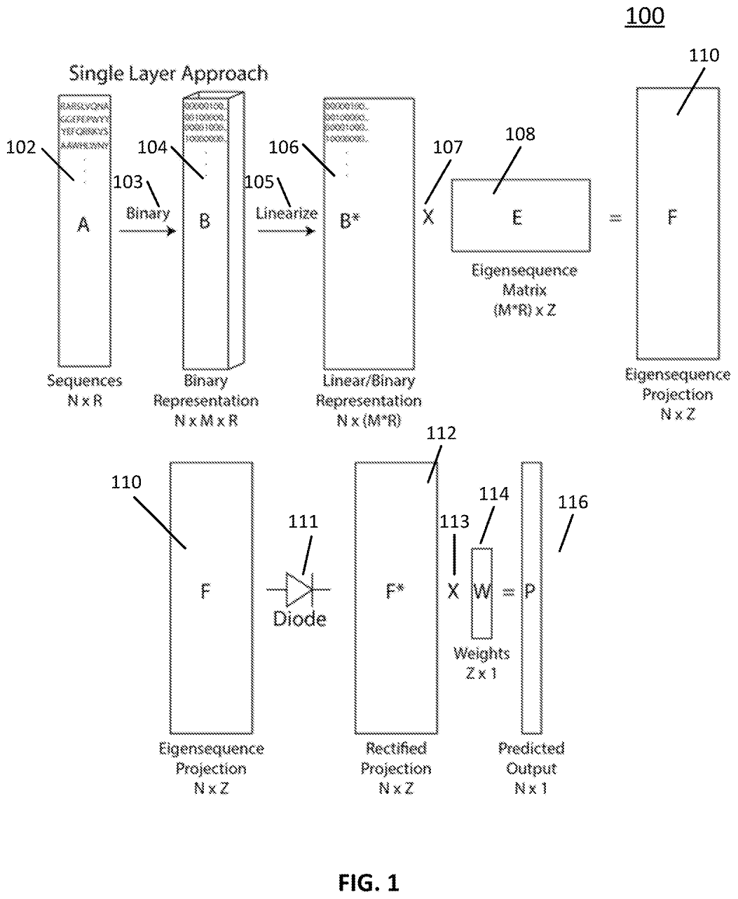

Note that, the techniques described herein describe use of a molecular library. In some embodiments, any suitable technique or combination of techniques can be used to prepare a molecular library, such as phage display, RNA display, synthetic bead-based libraries, other library techniques using synthesized molecules, and/or any other suitable technique(s). The techniques described herein are applicable to any molecular library system in which the function in question can be measured for enough of the unique molecular species in the library to allow the fitting routine (described below in more detail in connection with FIGS. 1-4) to properly converge.

Additionally, note that the mechanisms described herein are generally described as implemented using large peptide arrays. However, in some embodiments, any other suitable type of molecular library for which the structure of some or all of the molecules in the library can be described in terms of a common set of structural features, and a measured response associated with that structure, can be used. Other examples of molecular libraries which can be used include peptides, peptoids, peptide nucleic acids, nucleic acids, proteins, sugars and sugar polymers, any of the former with non-natural components (e.g., non-natural amino acids or nucleic acids), molecular polymers of known covalent structure, branched molecular structures and polymers, circular molecular structures and polymers, molecular systems of known composition created in part through self-assembly (e.g., structures created through hybridization to DNA or structures created via metal ion binding to molecular systems), and/or any other suitable type of molecular library. In some embodiments, the measured response can include binding, chemical reactivity, catalytic activity, hydrophobicity, acidity, conductivity, electromagnetic absorbance, electromagnetic diffraction, fluorescence, magnetic properties, capacitance, dielectric properties, flexibility, toxicity to cells, inhibition of catalysis, inhibition of viral function, index of refraction, thermal conductivity, optical harmonic generation, resistance to corrosion, resistance to or ease of hydrolysis, and/or any other suitable type of measurable response.

In some embodiments, the mechanisms described herein can use a neural network with any suitable type of architecture. In some embodiments, an input to the neural network can include information regarding a sequence of a heteropolymer, such as a peptide. In some embodiments, an output of the neural network can include an indication of a measurable function (e.g., binding, modification, structure, and/or any other suitable function). In some embodiments, information regarding a sequence that is used as an input to the neural network can be represented in any suitable format. For example, in some embodiments, as described below in more detail in connection with FIGS. 1-4, a vector-based representation can be used. As a more particular example, each amino acid in a sequence can be represented by a vector of some length based on its physical characteristics (e.g., charge, hydrophobicity, and/or any other suitable physical characteristics) and/or based on combinations of physical properties (e.g., principle components of sets of physical properties).

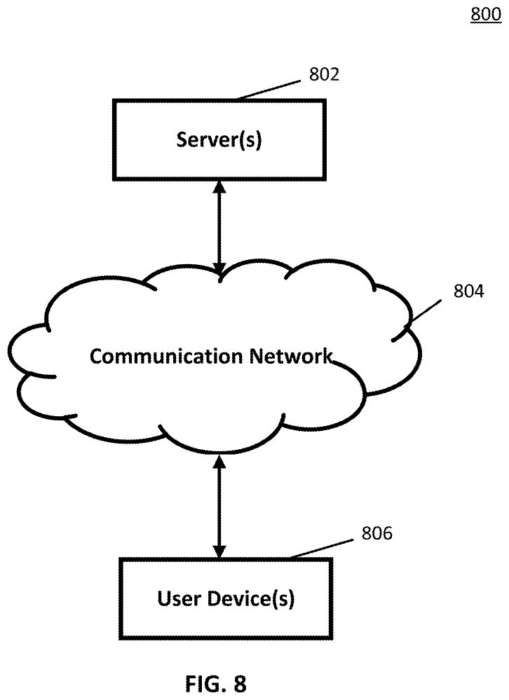

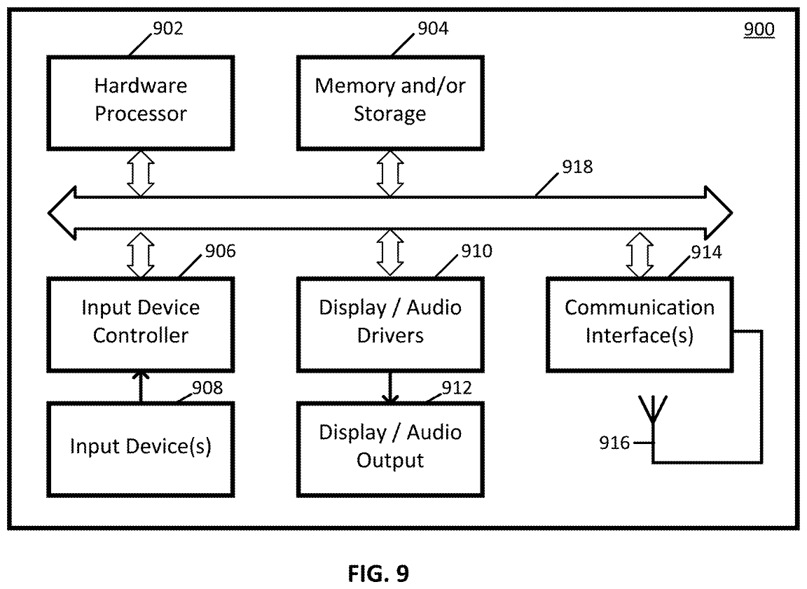

Turning to FIG. 1, an example of a process 100 for implementing a single-hidden-layer neural network for predicting functions of molecular sequences is shown in accordance with some embodiments of the disclosed subject matter. Note that, in some embodiments, process 100 can be implemented on any suitable device, such as a server and/or a user device (e.g., a desktop computer, a laptop computer, and/or any other suitable type of user device), as shown in and described below in connection with FIGS. 8 and 9.

Process 100 can begin by generating a peptide array 102 (e.g., array A as shown in FIG. 1) based on a sequence of peptides. As illustrated, in some embodiments, the number of peptides is N, and the number of residues per peptide is R. In some embodiments, N can have any suitable value (e.g., 1000, 10,000, 100,000, 200,000, 1,000,000, 5,000,000, 10,000,000, and/or any other suitable value). Note that, in some embodiments, variable peptide length can be accommodated by padding the shorter sequences with an unused character.

At 103, process 100 can generate a binary representation 104 for the peptide array (e.g., array B as shown in FIG. 1). For example, in some embodiments, an orthonormal vector description for each amino acid can be used. As a more particular example, if M is the number of different amino acids used on the array, then each vector describing an amino acid is M long and is all zeros except one element. In some embodiments, process 100 can generate a matrix representation for each peptide sequence that is of size M×R, and can generate a three-dimensional total binary array that is of size N×M×R.

Note that, although array A has generally been described herein as dividing peptides into amino acids, in some embodiments, process 100 can divide the peptides in any suitable manner. For example, in some embodiments, peptides can be divided based on connectivity of substituent groups (carboxylic acids, amines, phenyl rings) and/or in terms of individual atoms. As a more particular example, in some embodiments, a structure can be encoded within a vector hierarchically by following covalent bonding lines. Additionally or alternatively, in some embodiments, peptides can be divided based on amino acid pairs. Continuing with this example, in some embodiments, a binary vector can have M² bits for each residue. Furthermore, in some embodiments, molecular libraries do not have to be represented as arrays. For example, in some embodiments, bead libraries or other library approaches can be used.

At 105, process 100 can linearize array B 104 (that is, the binary representation of the molecular library) to produce linear/binary representation N×(M*R)B* 106. For example, in some embodiments, the matrix representation can be linearized such that binary descriptions of each amino acid in that peptide are concatenated end-to-end which can have size N×(M*R) (e.g., array B* as shown in FIG. 1).

At 107, process 100 can multiply the linearized matrix representation array B* 106 by an eigensequence matrix 108 (e.g., array E as shown in FIG. 1) to produce eigensequence projection F 110. In this case, eigensequence matrix 108 is a hidden layer of the neural network. In some embodiments, the eigensequence matrix can have Z columns, which can represent a sequence space. In the particular example shown in FIG. 1, the eigensequence matrix can be of size (M*R)×Z. In some embodiments, each column of the eigensequence matrix can be thought of as a conceptual peptide sequence. For example, in some embodiments, instead of being M−1 zeros and one 1, the eigensequence matrix can be a real-valued system that represents a mixed peptide. In some embodiments, the mixed peptide or eigensequence can be eigensequences that are required to describe the space accurately. In some embodiments, the number of eigensequences can reflect the number of distinct kinds of sequence dependent function that are resolved in the system. For example, in an instance of binding of a protein to the peptide array, this might be the number of different (though potentially overlapping) sites involved in the binding.

In some embodiments, eigensequence projection matrix F 110 (e.g., matrix F as shown in FIG. 1) can be a projection of each of the N peptides onto the axes of the sequence space defined by the Z eigensequences. As illustrated, in some embodiments, matrix F 110 can have a size of N×Z.

At 111, process 100 can apply an activation function to eigensequence projection matrix F 110 to generate a rectified matrix 112 (e.g., matrix F* as shown in FIG. 1). In some embodiments any suitable activation function can be used, such as a perfect diode activation function, as illustrated in FIG. 1. In some embodiments, the perfect diode activation function shown in FIG. 1 can act like a feature selection process, effectively removing the contribution of any eigensequence below a threshold. Note that, in some embodiments, any other suitable type of activation function can be used. Additionally note that while there is no bias applied to the system as shown in FIG. 1 (e.g., subtracting a set bias value from matrix F), in some embodiments, a positive bias can be applied to increase the stringency of the feature selection process. In some embodiments, applying a positive bias can cause the algorithm to consider only the most important eigensequence projections for any particular peptide sequence.

At 113, process 100 can multiply the rectified matrix F* 112 by a final weighting function 114 (e.g., vector W as shown in FIG. 1) to produce predicted output P 116. As illustrated, vector W can have size Z×1. In some embodiments, the weighting function can provide a weighting value for the projections on each of the Z eigensequences for each of the N peptide sequences. In some embodiments, predicted output P 116 can be a predicted functional output for each of the sequences, as shown by vector P in FIG. 1. In some embodiments, vector P can have size N×1.

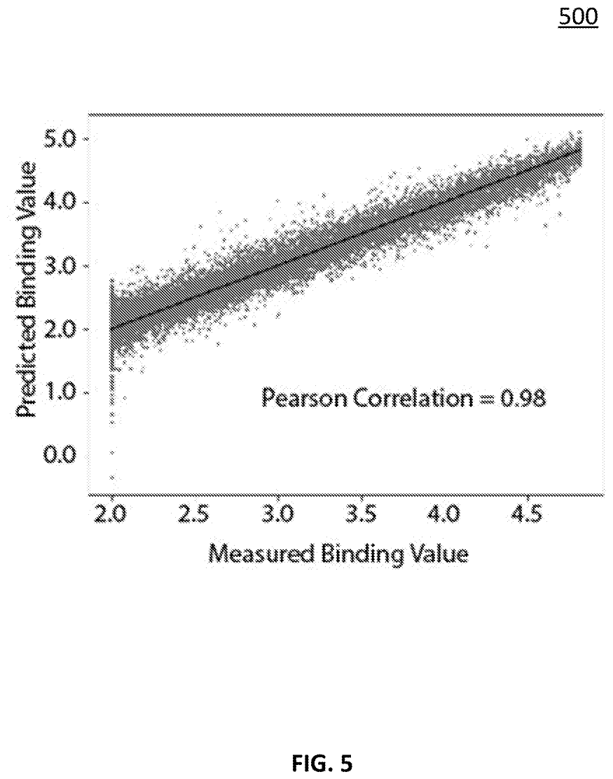

In some embodiments, any suitable technique or combination of techniques can be used to train the neural network described above. For example, in some embodiments, matrices E and W can be determined using any suitable nonlinear optimization technique(s) (e.g., gradient descent, stochastic descent, conjugated gradient descent, and/or any other suitable technique(s)). Additionally, note that, in some embodiments, any suitable training set of any suitable size can be used, as described in more detail below in connection with FIGS. 5-7. In some embodiments, test sequences not involved in the training can be used as inputs and evaluated based on their known outputs. In some such embodiments, a degree of overfitting can be determined based on prediction of training set values and prediction of test set values. Note that, in some embodiments, any suitable software libraries or packages can be used to implement the neural network (e.g., Google TensorFlow, PyTorch and/or any other suitable software libraries or packages).

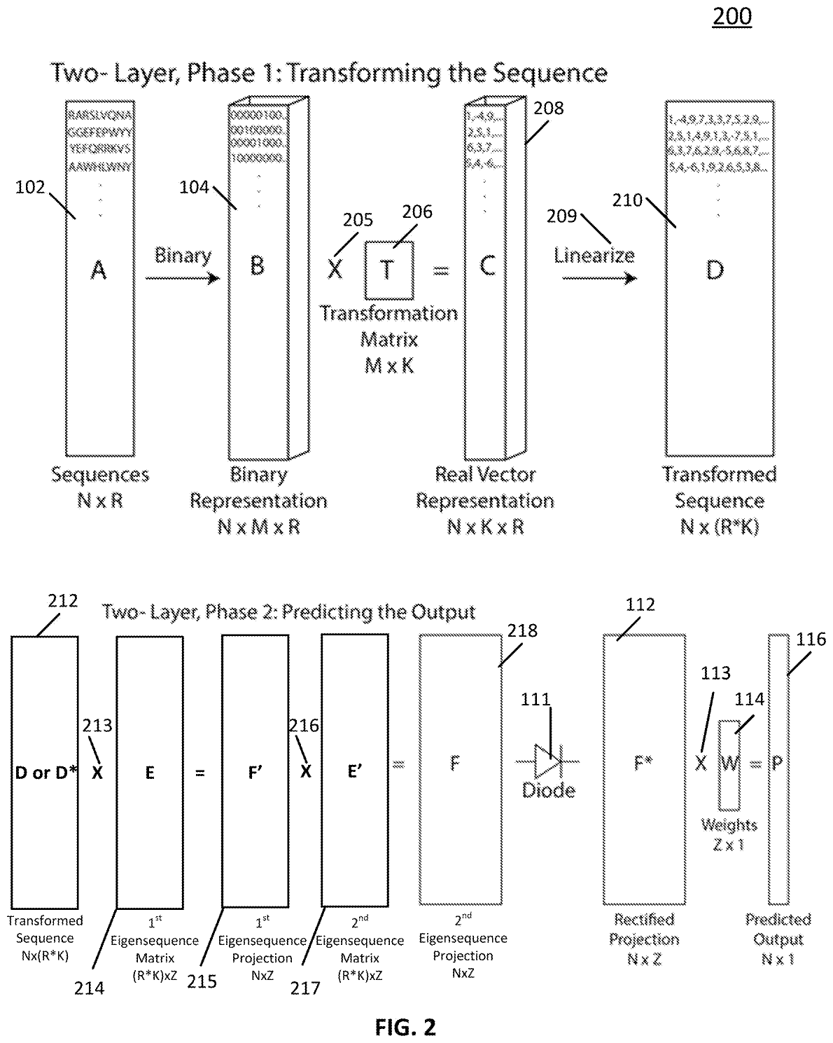

Turning to FIG. 2, an example 200 of a process for implementing a two-hidden-layer neural network for predicting functions of molecular sequences is shown in accordance with some embodiments of the disclosed subject matter. In some embodiments, additional layers can be added to the neural network to increase the nonlinearity of the network, or to divide the network up into physically meaningful components. Note that, in some embodiments, blocks of process 200 can be implemented on any suitable device, such as a server and/or a user device (e.g., a desktop computer, a laptop computer, and/or any other suitable type of user device), as shown in and described below in connection with FIGS. 8 and 9.

In some embodiments, process 200 can begin similarly to what is described above in connection with FIG. 1, by generating matrices A 102 and B 104. In some embodiments, process 200 can then, at 205, transform the binary vectors of matrix B 104 into a real valued matrix C 208, such that each amino acid now has a specific real-valued vector of length K characterizing it. In some embodiments, process 200 can generate matrix C by multiplying a transformation matrix 206 (e.g., matrix T as shown in FIG. 2) by each binary matrix representing the sequence (e.g., matrix B as shown in FIG. 2). In some embodiments, the transformation matrix T can be a set of values describing each amino acid in the system and can be of size M×K. This is often useful in determining, for example, if two similar amino acids are both needed in the design of the peptide array (e.g., to determine a similarity and/or a degree of similarity between two amino acids, such as glutamate and aspartate).

At 209, process 200 can then linearize matrix C 208 to generate matrix D 210 using the techniques described above in connection with 105 of FIG. 1.

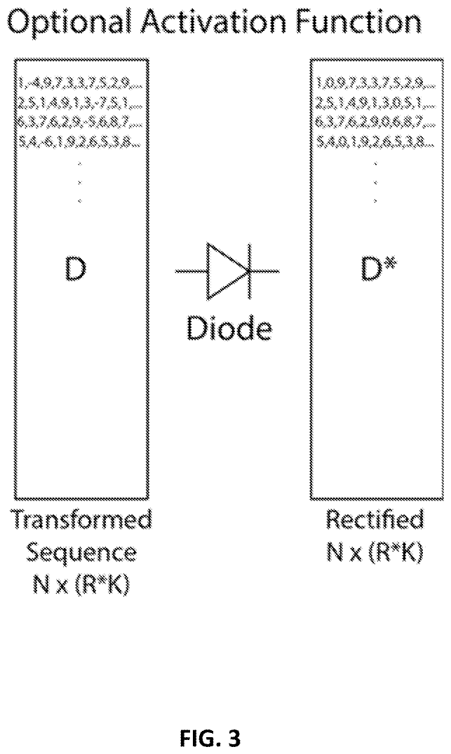

Note that, in some embodiments, process 200 can add a nonlinear step after the linearization at 209. For example, in some embodiments, process 200 can apply an activation function to matrix D. As a more particular example, process 200 can apply an activation function (e.g., a rectifier, and/or any other suitable activation function) to matrix D to generate matrix D*, as shown in FIG. 3. In some embodiments, an activation function can be used to generate better predictions using fewer parameters. Conversely, in some embodiments, not applying an activation function to matrix D can allow a description of the amino acids to be separated from a description of the eigensequences.

At 213, matrix D or D* 212 (which matrix 212, when matrix D, can be the same as matrix D 210) can be multiplied by 1st eigensequence matrix 214 (e.g., matrix E as shown in FIG. 2), similarly to what was described above in connection with 107 of FIG. 1 to generate a 1st eigenspace projection 215 (e.g., matrix F′ as shown in FIG. 2). In some embodiments, the size of the matrix D can depend on K, rather than M. In this case, eigensequence matrix 214 is a first hidden layer of the neural network.

At 216, matrix F′ 215 can be multiplied by 2nd eigensequence matrix 217 (e.g., matrix E′ as shown in FIG. 2), similarly to what was described above in connection with 107 of FIG. 1 to generate a 2nd eigenspace projection 215 (e.g., matrix F′ as shown in FIG. 2). In this case, eigensequence matrix 217 is a second hidden layer of the neural network.

In some embodiments, process 200 can then apply any suitable activation function 111 to provide matrix F* 112 and then apply weights 114 at 113 to matrix F* 112 to generate a predicted output 116 (e.g., vector P as shown in FIG. 2), similarly to what was described above in connection with blocks 110 and 112 of FIG. 1.

In some embodiments, process 200 can determine matrices T, E, E′, and W using any suitable nonlinear optimization techniques, similarly to what was described above in connection with FIG. 1. In some embodiments, the T, E, E, and W matrices can be used for any suitable purposes. For example, if one is making a molecular recognition array to interrogate some function, one often wants to know the minimum number of monomers (different amino acids in the case of peptide arrays) that are required to capture the desired functional information. The rows of the T array are the amino acid descriptions. One can ask the question whether each row in the matrix is mathematically independent of all the other rows; how well can each row be represented by a linear combination of the others? This gives quantitative information about which amino acids are most unique in the description. As another example, in the E and E matrices, one is more interested in the columns which really represent a set of sequences that are being used to describe the sequence/function space. Simply decreasing that number to a minimum required for adequate description (particularly if no rectification is done after creation of the D matrix), that is, to a number of molecular recognition eigenvectors that are required to describe the function, can be useful information. For binding, that is likely closely related to the number of sequence specific sites that behave differently from one another. Again, for minimizing the cost and complexity of molecular array production, zeroing in on a small set of sequences most similar to the eigensequence representations could be very beneficial. In principle, one can ask what is the minimum number of real sequences (after transformation by T) required to provide a complete description of the set of eigensequences.

Note that, in some embodiments, any other suitable number of hidden layers can be added to the neural network.

While eigensequence matrices 108, 214, and 217 are described herein as being used as hidden layers of the neural networks, any other suitable form of hidden layer can be used in some embodiments.

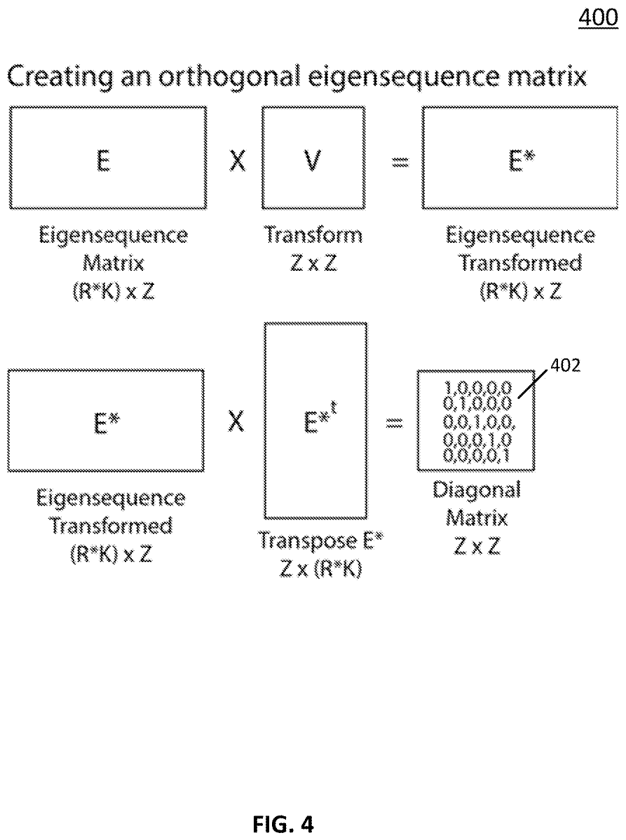

In some embodiments, an eigensequence (e.g., matrices E and E as shown in FIGS. 1 and 2) can be orthonormalized in any suitable manner. For example, in some embodiments, a neural network as shown in FIGS. 1 and/or 2 can orthonormalize matrices E and E. FIG. 4 shows an example 400 of a process for orthonormalizing an eigensequence.

In some embodiments, process 400 can add or subtract any column in matrix E or E from any other column in matrix E or E to create a new set of eigensequences describing the same space but having rotated vectors. In some embodiments, process 400 can identify a transformation matrix V (as shown in FIG. 4) that converts matrix E to matrix E* such that E*xE*^{t} is a unity matrix 402. In some such embodiments, processes 100 and/or 200 can additionally adjust weight vector W, as described above in connection with FIGS. 1 and/or 2.

In some embodiments, in instances where process 400 is used in conjunction with processes 100 and/or 200, processes 100 and/or 200 can iterate between process 400 and adjusting matrix E (e.g., using a nonlinear optimization algorithm as described above in connection with FIGS. 1 and 2).

In some embodiments, processes 100, 200, and/or 400 can be used for any suitable applications. For example, in some embodiments, a neural network as described above can be trained and/or optimized to predict the binding of peptide sequences to a particular protein of interest. In some such embodiments, the processes described above can be used to search for potential protein partners in the human proteome. For example, in some embodiments, the sequences of the proteome can be tiled into appropriately sized sequence fragments and can be used to form matrix A as shown above in connection with FIGS. 1 and/or 2. Tests with a Xeon, 20 core desktop processor have shown that it is possible to scan the human proteome in seconds. Indeed, it is possible to predict binding of all possible 9-mer peptides (˜5×10¹¹ peptides) in a few days, a fact that can be very useful for ligand design projects.

Note that, in some embodiments, a molecular library can be assayed for function. For example, a function can be binding to a specific target (small molecule, protein, material, cells, pathogens, etc.), chemical reactivity, solubility, dynamic properties, electrical properties, optical properties, toxicity, pharmacokinetics, effects on enzymes, effects on cells (e.g., changes in gene expression or metabolism), effects on pathogens or any other effect that can be measured on the whole, or a large fraction, of the library resulting in a quantitative value or a qualitative result that can be represented as one of two or more alternatives. In some embodiments, in the case of binding interactions, targets can be labeled directly or indirectly or label free approaches can be used to detect binding. In some embodiments, isolated target binding can be considered alone or can be compared to target binding in the presence of a known binding ligand or other biomolecule. In the latter case, an aspect of the binding pattern due to the interaction with the ligand or the biomolecule can be identified, thereby allowing a known drug and its known target and to be used to generate a new ordered molecular component arrangement that mimics the binding of the drug.

Additionally, note that, in some embodiments, analyzing a function, such as binding, for a sparse sampling of particular ordered combinations of molecular components to form larger structures can be used to predict the function for all ordered combinations with similar structural characteristics (same set of molecular components, same kind or kinds of bonds, same kind of overall structure such as linear sequence, circular sequence, branched sequence, etc.). In some embodiments, a general quantitative relationship between the arrangement and identity of molecular components and the function can be derived using any suitable approach(es), including using a basis set of substructures and appropriate coefficients or using machine learning approaches.

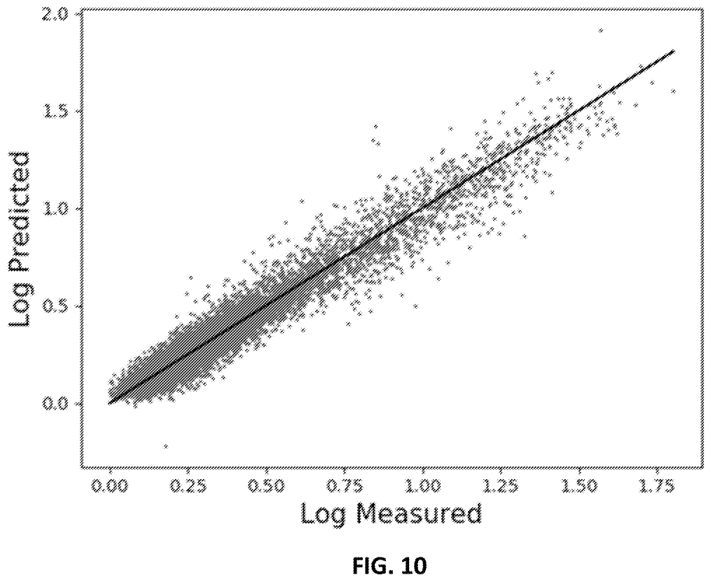

In some embodiments, after a parameterized fit that describes a function of ordered combinations of molecular components in terms of some function(s) has been generated, the resulting parameterized fit can be used to optimize the ordered combination around the function(s) such that one or more new ordered combinations can be generated that are not in the original molecular library and are predicted to have functions that are more appropriate for the application of interest than any molecules in the original molecular library. In some embodiments, one or more optimized molecules (e.g., identified based on the one or more new ordered combinations) can be synthesized, and the function can be verified, as described below in more detail in connection with FIGS. 10-13.

Specific applications and their results are described below in connection with FIGS. 5-7 and FIGS. 10-13.

Note that, in the specific applications described below in connection with FIGS. 5-7, peptide arrays can be exposed to individual antibodies, serum containing antibodies, or to specific proteins. In some embodiments, antibodies or other proteins can bind to the array of peptides and can be detected either directly (e.g., using fluorescently labeled antibodies) or by the binding of a labeled secondary antibody. In some embodiments, the signals produced from binding of the target to the features in the array form a pattern, with the binding to some peptides in the array much greater than to others. In some embodiments, a peptide array can include any suitable number of peptides. For example, the specific arrays described below in connection with FIGS. 5-7 included between 120,000 and 130,000 unique peptides, although larger and smaller sized libraries can be used.

It should be noted that the arrays used in these applications have been extensively employed not only for antibody and protein binding but for binding to small molecules, whole viruses, whole bacteria and eukaryotic cells as well. See, e.g., Johnston, Stephen & Domenyuk, Valeriy & Gupta, Nidhi & Tavares Batista, Milene & C. Lainson, John & Zhao, Zhan-Gong & Lusk, Joel & Loskutov, Andrey & Cichacz, Zbigniew & Stafford, Phillip & Barten Legutki, Joseph & Diehnelt, Chris, “A Simple Platform for the Rapid Development of Antimicrobials,” Scientific Reports, 7, Article No. 17610 (2017), which is hereby incorporated by reference herein in its entirety. In some embodiments, functions other than binding such as chemical modification (e.g., phosphorylation, ubiquination, adenylation, acetylation, etc.), hydrophobicity, structure response to environmental change, thermal conductivity, electrical conductivity, polarity, polarizability, optical properties (e.g., absorbance, fluorescence, harmonic generation, refractive index, scattering properties, etc.) can be measured and modeled. The analysis described applies to all of these cases. Array synthesis and binding assays in the examples given below were performed as has been described in the literature. See, e.g., Legutki J B, Zhao Z G, Greying M, Woodbury N, Johnston S A, Stafford P, “Scalable High-Density Peptide Arrays for Comprehensive Health Monitoring,” Nature Communications, 5, 4785. PMID: 25183057 (2014), which is hereby incorporated by reference herein in its entirety. For some of the studies, the arrays were synthesized and or assays performed by the company HealthTell, Inc., of San Ramon, Calif., (www.healthtell.com). For other studies the arrays were synthesized and/or assays performed in the Peptide Array Core (www.peptidearraycore.com) at Arizona State University.

Turning to FIG. 5, an example is shown of using the approach described above in connection with FIGS. 1-4 to predict a binding value of the protein transferrin to an array of ˜123,000 peptides. Approximately 110,000 peptides were used to train a neural network with matrices of the following dimensions: T=16×7; E=91×200 (as the maximum length of peptides as 13, and 7×13=91); and W=200×1. Note that more detailed descriptions of the matrices are given above in connection with FIGS. 1-4. In the case of the dataset used to produce the results of FIG. 5, training was done in such a way that the entire range of binding values was equally weighted (the algorithm involves sampling a subset of binding values at each iteration of the fit and this was done in such a way that sampling always used an even distribution across the range of values). However, note that, in some embodiments, even sampling is simply one approach to data weighting, and any suitable data weighting technique(s) can be applied.

FIG. 5 shows an example of the application of a trained neural network model (as described above in connection with FIGS. 1-4) to ˜13,000 peptides that were held out of the training. As illustrated, the correlation coefficient of the measured to predicted values is 0.98. Note that two datasets were averaged to produce the data analyzed in this fit. The correlation between those two datasets was a little over 0.97. The average of the two should have removed a fraction of the noise and thus 0.98 is approximately what one would expect for a fit that has extracted all possible information from the dataset relative to the inherent measurement noise.

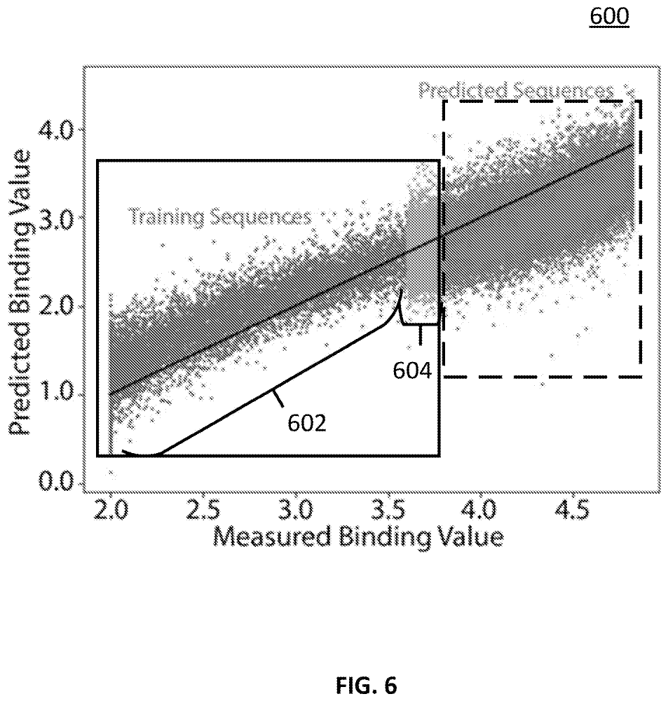

Turning to FIG. 6, another example of using the approach described above in connection with FIGS. 1-4 to predict a binding value of the protein transferrin to an array of peptides is shown in accordance with some embodiments. For the training set used to produce the results of FIG. 6, training of the neural network was completed using peptides with low binding values (indicated as “training sequences” and highlighted with a solid line in FIG. 6) and the testing set used peptides with high binding values (indicated as “predicted sequences” and highlighted with a dashed line in FIG. 6). Thus, the algorithm extrapolated from low binding values used for training the neural network to high binding values used in the test set. To do this most efficiently, the training data was separated into two parts, as shown in the figure (a first portion 602 and a second portion 604). The bulk of the training took place using first portion 602, but at the end, the training was continued for additional iterations, selecting the iteration that best described second portion 604. The model that arose more consistently allows appropriate representation of the test data (indicated as “predicted sequences” and highlighted with a dashed line) than does a fit without this final model biasing step.

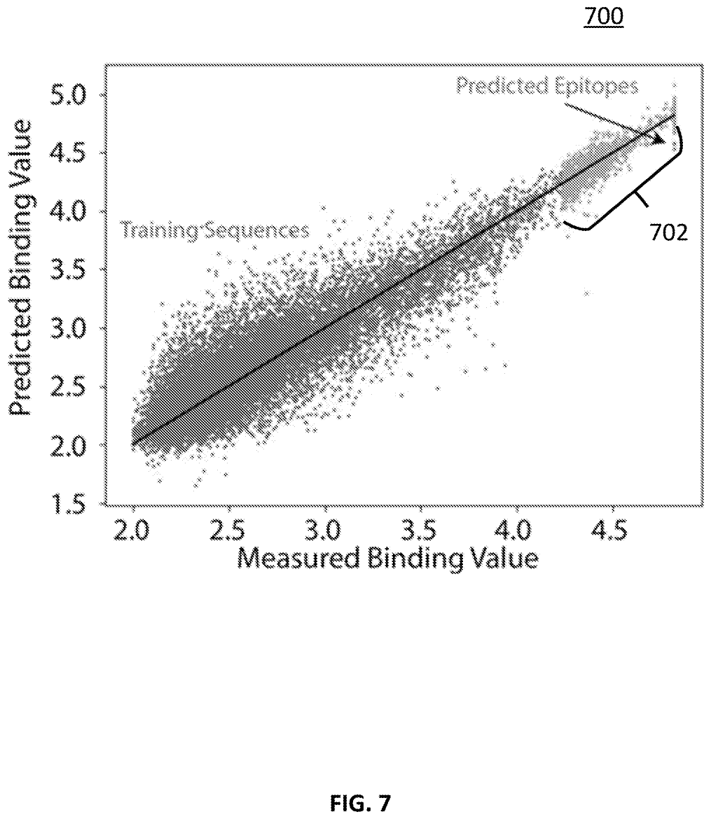

Turning to FIG. 7, an example of using the approach described above in connection with FIGS. 1-4 to predict the cognate epitope of a monoclonal antibody is shown in accordance with some embodiments of the disclosed subject matter. DM1A is a monoclonal antibody for alpha tubulin (raised to chicken, but is often used for human), and the cognate epitope is AALEKDY. The peptide arrays used for these studies contains this epitope as well as ALEKDY. To produce the results shown in FIG. 7, both of these sequences were removed from the training data and the algorithm was used to predict their values. A training technique similar to what was described above in connection with FIG. 6 was used to train the neural network. In particular, training was split into two parts, a first part that involved the normal fit process and the highest values that were used to select the final fit used in the extrapolation. The results of this analysis are shown in FIG. 7. As illustrated, cognate sequences 702 are among the highest binding peptides.

Note that, although the examples described herein generally relate to measuring binding, in some embodiments, any other suitable type of function can be used. For example, in some embodiments, any suitable function can be used for which the function can be measured for each type of molecule in the molecular library. Specific examples of functions can include chemical reactivity (e.g., acid cleavage, base cleavage, oxidation, reduction, hydrolysis, modification with nucleophiles, etc.), enzymatic modification (for peptides, that could be phosphorylation, ubiquination, acetylation, formyl group addition, adenylation, glycosylation, proteolysis, etc.; for DNA, it could be methylation, removal of cross-linked dimers, strand repair, strand cleavage, etc.), physical properties (e.g., electrical conductivity, thermal conductivity, hydrophobicity, polarity, polarizability, refraction, second harmonic generation, absorbance, fluorescence, phosphorescence, etc.), and/or biological activity (e.g., cell adhesion, cell toxicity, modification of cell activity or metabolism, etc.).

Molecular recognition between a specific target and molecules in a molecular library that includes sequences of molecular components linked together can be comprehensively predicted from a very sparse sampling of the total combinatorial space (e.g., as described above in connection with FIGS. 1-4). Examples of such sparse sampling and quantitative prediction are shown in and described below in connection with FIGS. 10-13.

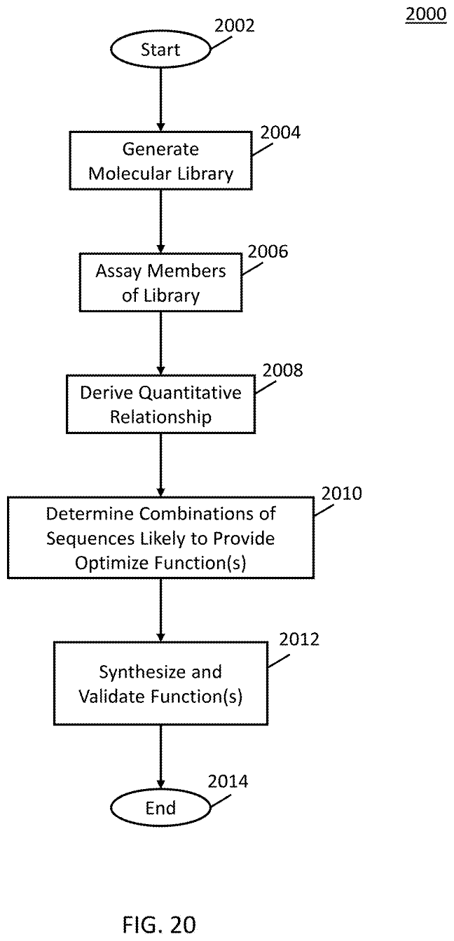

Note that, in the examples shown in and described below in connection with FIGS. 10-13, the results can be generated using an example process 2000 as illustrated in FIG. 20. As shown, after process 2000 begins at 2002, the process can, at 2004, generate a molecular library. Any suitable molecular library can be generated in some embodiments. For example, in some embodiments, the molecular library can include a defined set of molecular components in many ordered combinations linked together by one or a small number of different kinds of chemical bonds.

Next, at 2006, the process can assay the members of the molecular library for a specific function of interest. In some embodiments, the members can be assayed in any suitable manner and for any suitable function of interest in some embodiments.

Then, at 2008, the process can derive a quantitative relationship between the organization or sequence of the particular combination of molecular components for each member of the library to function(s) or characteristic(s) of that combination using a parameterized fit(s). In some embodiments, any suitable quantitative relationship can be derived and the quantitative relationship can be derived in any suitable manner.

At 2010, process 2000 can then determine combinations of sequences likely to provide optimized function(s). The combinations of sequences can be determined in any suitable manner in some embodiments. For example, in some embodiments, process 2000 can use the parameterized fit(s) to determine, from a larger set of all possible combinations of molecular components linked together, combinations of sequences likely to provide optimized function(s).

Then, at 2012, process 2000 can synthesize and empirically validate the function(s). In some embodiments, the functions can be synthesized and empirically validated in any suitable manner.

Finally, process 2000 can end at 2014.

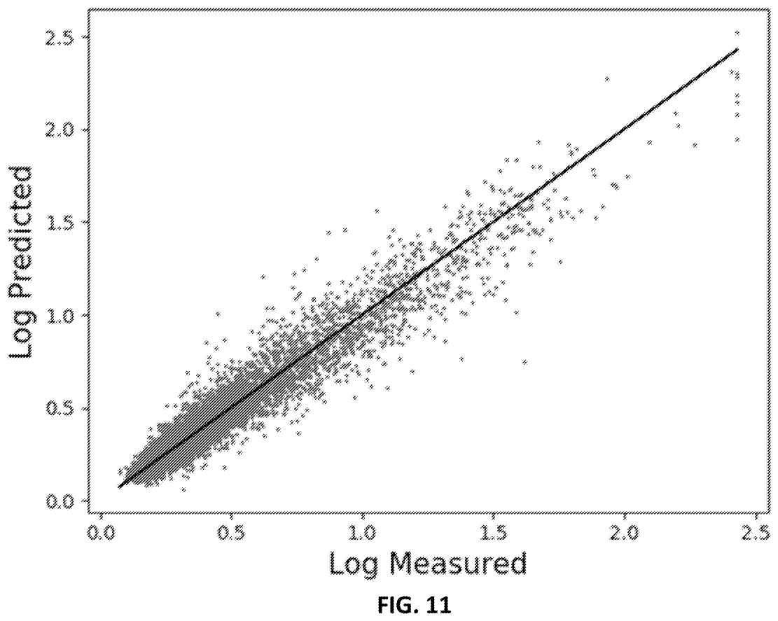

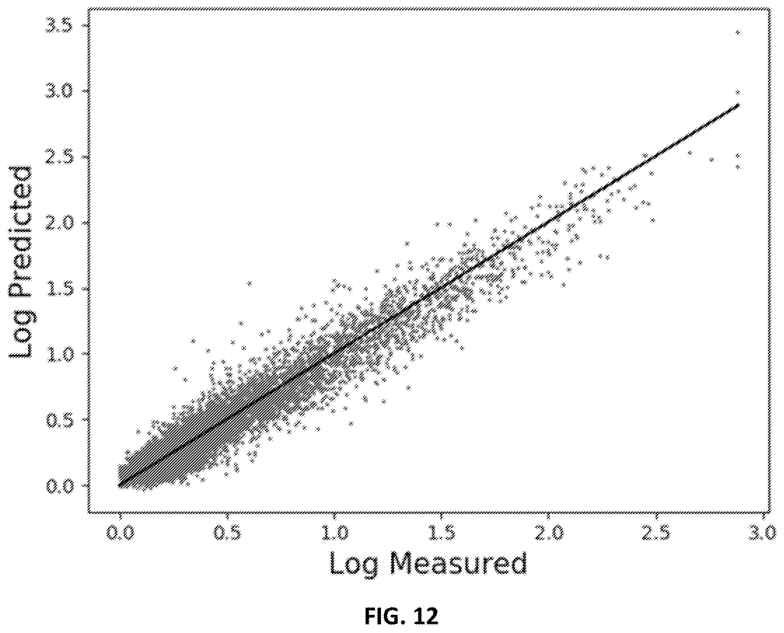

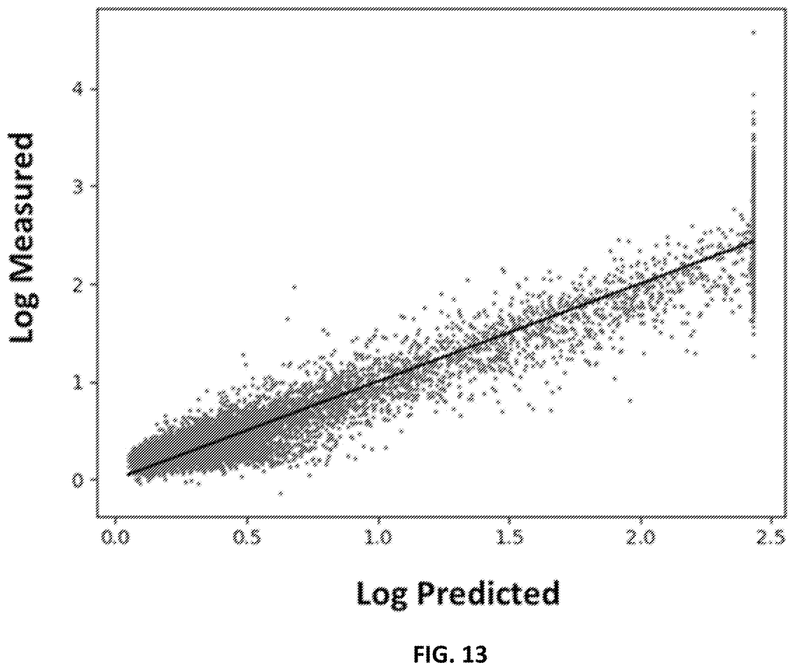

Turning to FIGS. 10 and 11, results of binding PD1 and PDL1 (natural binding partners), respectively, to an array of ˜125,000 unique peptide sequences and fitting a relationship between the peptide sequence and the binding values using a machine learning algorithm shown in FIG. 1 are shown. The resulting relationship can then be applied to any peptide sequence to predict binding. On a Xeon processor with 20 cores, 10¹² sequences (e.g., all possible peptide sequences with 9 residues) can be considered in a few days, and both the binding strength and binding specificity of all sequences can be determined. Alternatively, a binding profile can be derived for the complex between PD1 and PDL1. Peptides that bind to one or the other, but not to the complex, likely will interfere with complex formation. The measurement can be performed on arrays made with non-natural amino acids, such as D-amino acids, or with completely different molecular components that may or may not be amino acids. This can provide final compounds with greater stability and better pharmacokinetics when applied in vivo. Note that FIGS. 12 and 13 show similar results for TNFα and one of its receptors, TNFR2. A similar analysis and application of the resulting relationships could be used to find sequences to bind to one or the other or that would interfere with the binding of one to the other. PD1, PDL1, and TNFα are all targets of highly successful drugs.

In the examples of FIGS. 10 and 11, each of the molecules in the library is attached via a base-cleavable linker to a surface and has a charged group on one end as described in Legutki, J. B.; Zhao, Z. G.; Greying, M.; Woodbury, N.; Johnston, S. A.; Stafford, P., “Scalable High-Density Peptide Arrays for Comprehensive Health Monitoring,” Nat Commun 2014, 5, 4785, which is hereby incorporated by reference herein in its entirety. The library is exposed to freshly prepared whole blood and incubated at body temperature for 3 hours and then extensively washed to remove all possible material other than the library molecules. The linker is cleaved using ammonia gas and the mass spectrum of the resulting compounds are determined via matrix assisted laser desorption ionization mass spectrometry (see Legutki et al.). This is compared to a control in which the sample was not exposed to blood. The relative proportion of the mass spectrum that includes the desired peak for each molecule in the library is then determined quantitatively for both the blood exposed and unexposed libraries. A relationship between the sequence and the relative survival of the compound to exposure to blood is determined by fitting as in the examples above. Using an equation derived from the relationship between the sequence and the relative survival of the component to exposure to blood, compounds determined using the equations and the relationships shown in FIGS. 10 and 11 are screened for their predicted stability in whole blood.

Referring to FIG. 10, fitting results using the binding pattern of the extracellular portion of the protein PD1 (programmed death 1) to a peptide array with ˜125,000 unique peptide sequences that were chosen to cover sequence space evenly, though sparsely, are shown. The peptides averaged 9 residues in length and included 16 of the 20 natural amino acids (A,D,E,F,G,H,L,N,P,Q,R,S,V,W,Y). ˜115,000 of the sequences and the binding intensities from the array were used to train a neural network (e.g., as shown in and described above in connection with FIG. 1). The resulting equation was then used to predict the binding of the remaining 10,000 peptide sequences. FIG. 10 shows the predicted values of the sequences not used in the fit versus the measured values.

Referring to FIG. 11, fitting results using the binding pattern of the extracellular portion of the protein PDL1 (programmed death ligand 1) to a peptide array with ˜125,000 unique peptide sequences that were chosen to cover sequence space evenly, though sparsely, are shown. The peptides averaged 9 residues in length and included 16 of the 20 natural amino acids (A,D,E,F,G,H,L,N,P,Q,R,S,V,W,Y). ˜115,000 of the sequences and the binding intensities from the array were used to train a neural network (e.g., as shown in and described above in connection with FIG. 1). The resulting equation was then used to predict the binding of the remaining ˜10,000 peptide sequences. The figure shows the predicted values of the sequences not used in the fit versus the measured values.

Referring to FIG. 12, fitting results using the binding pattern of the protein TNFα (tumor necrosis factor alpha) to a peptide array with ˜125,000 unique peptide sequences that were chosen to cover sequence space evenly, though sparsely, are shown. The peptides averaged 9 residues in length and included 16 of the 20 natural amino acids (A,D,E,F,G,H,L,N,P,Q,R,S,V,W,Y). ˜115,000 of the sequences and the binding intensities from the array were used to train a neural network (e.g., as shown in and described below in connection with FIG. 1). The resulting equation was then used to predict the binding of the remaining ˜10,000 peptide sequences. FIG. 12 shows the predicted values of the sequences not used in the fit versus the measured values.

Referring to FIG. 13, fitting results using the binding pattern of the extracellular portion of the protein TNFR2 (TNFα receptor 2) to a peptide array with ˜125,000 unique peptide sequences that were chosen to cover sequence space evenly, though sparsely, are shown. The peptides averaged 9 residues in length and included 16 of the 20 natural amino acids (A,D,E,F,G,H,L,N,P,Q,R,S,V,W,Y). ˜115,000 of the sequences and the binding intensities from the array were used to train a neural network (e.g., as shown in and described above in connection with FIG. 1). The resulting equation was then used to predict the binding of the remaining ˜10,000 peptide sequences. FIG. 13 shows the predicted values of the sequences not used in the fit versus the measured values.

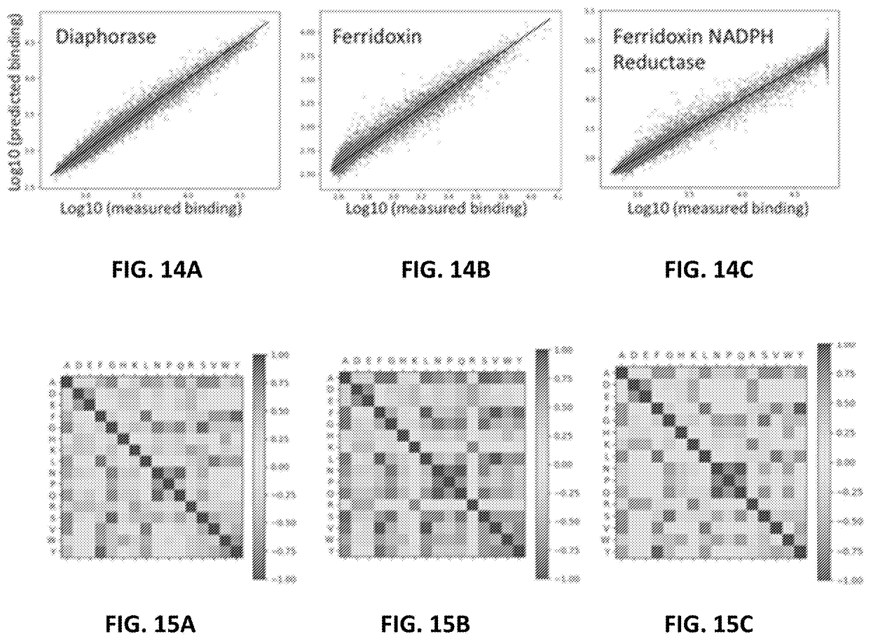

In conjunction with FIGS. 14A-19, examples of experiments used to test neural networks in accordance with some embodiments are now described.

Micromolar concentrations of three different fluorescently labeled proteins, diaphorase, ferredoxin and ferredoxin-NADP reductase, were incubated with separate arrays of ˜125000 peptides in standard phosphate saline buffer. The fluorescence due to binding of each protein to every peptide in each array was recorded. The experiment was performed in triplicate and the values averaged. The Pearson correlation coefficient between replicates for each protein was 0.98 or greater. 90% of the peptide/binding value pairs from each protein were used to train a neural network similar to that in FIG. 2. Each hidden layer (E and E in FIG. 2) in the neural network had a width of 100. The width of the T matrix in FIG. 2 was 10 (ten descriptors for each amino acid). The resulting trained network was used to predict the binding values for the remaining 10% of the peptide binding values. FIGS. 14A-14C shows scatter plots of the predicted versus the measured values for the 10% of the peptide/binding value pairs that were not involved in training the network. Both axes are in log base 10, so a change of 1 corresponds to a change of 10-fold in binding value. The Pearson Correlation Coefficients in each case are approximately the same as the correlation coefficients between technical replicates implying that the prediction is approximately as accurate as the measurement in each case. Increasing the number of hidden layers (E) in the neural network or increasing the size of the hidden layers (the number of values used in each transformation) does not appreciably improve the prediction.

FIGS. 15A-15C show examples of similarity matrices between the amino acids used to construct the peptides on the arrays in FIGS. 14A-14C, respectively. These similarity matrices were constructed by taking each column in matrix T of FIG. 2 and treating it as a vector. Note that each column corresponds to a particular amino acid used. Normalized dot products were then performed between these vectors, resulting in the cosine of the angle between them. The closer that cosine is to 1.0, the more similar the two amino acids. The closer the cosine is to 0.0, the less similar the amino acids. Negative values imply that there are dimensions in common, but that two amino acids point in opposite directions (e.g., E or D which have negative charges compared to K (lysine) or R (arginine) which have positive charges). In each case, there are strong similarities between amino acids D (aspartic acid) and E (glutamic acid) as well as between F (phenylalanine) and Y (tyrosine) and between V (valine) and L (leucine). This is what one would expect, as chemically these pairs of amino acids are similar to each other.

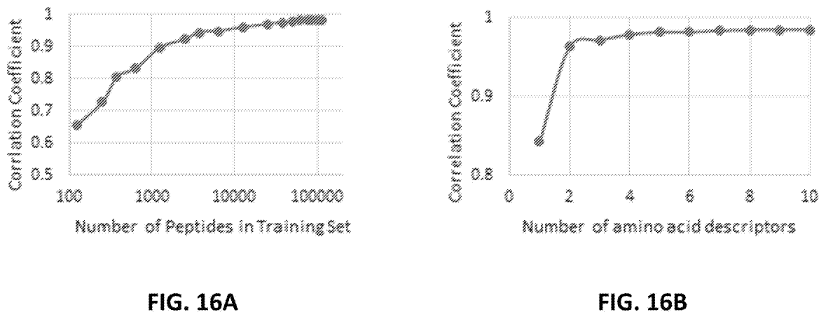

FIG. 16A shows an example of the Pearson Correlation between predicted and observed binding data as a function of the size of the training set in the above example. More particularly, this figure shows how the correlation coefficient for the predicted versus measured values of diaphorase changes as a function of the number of peptide/binding value pairs used in the training set. Interestingly, one finds that the correlation between predicted and measured is above 0.9 down nearly to the point of using only 1000 training values, suggesting that the topology of the binding space is smooth.

FIG. 16B shows an example of the Pearson Correlation between predicted and observed as a function of the number of descriptors used by the neural network to describe each amino acid. Again, surprisingly even just 3 descriptors give a relationship that is only slightly worse in terms of correlation than the best (7-8 descriptors).

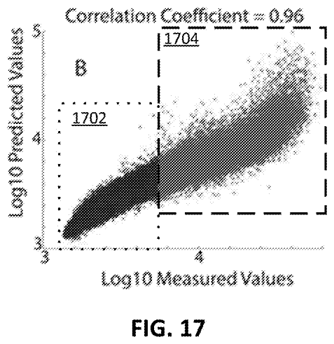

FIG. 17 shows an example of predicted versus measured values for diaphorase training only on weak binding peptides (box 1702) and predicting the strong binders (box 1704). Note that the axis scales are log base 10 of binding so the extrapolation takes place over more than an order of magnitude. This implies that the approach should also be amenable to binding prediction well beyond the dynamic range of the training data.

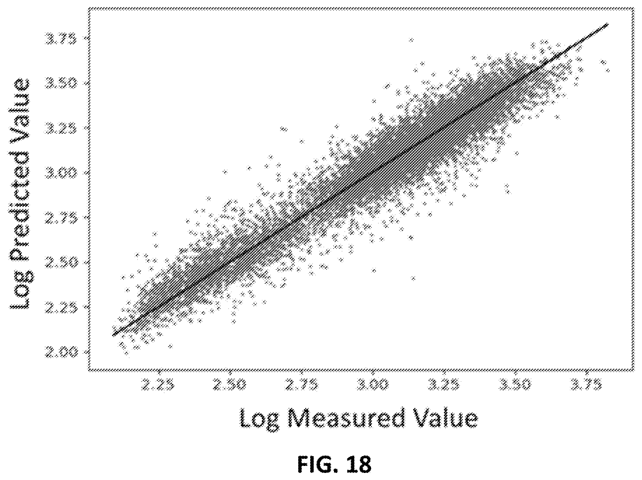

FIG. 18 shows an example of a prediction of the ratio between diaphorase binding and binding to total serum protein depleted of IgG, and demonstrates that the neural network can accurately predict specific binding to a particular protein (diaphorase in this figure). Here the binding values for diaphorase were divided by the binding values from an array incubated with a mix of labeled serum proteins (serum depleted of immune globlin G, IgG). Thus any aspect of the binding that is dominated by nonspecific binding to proteins in general would be eliminated.

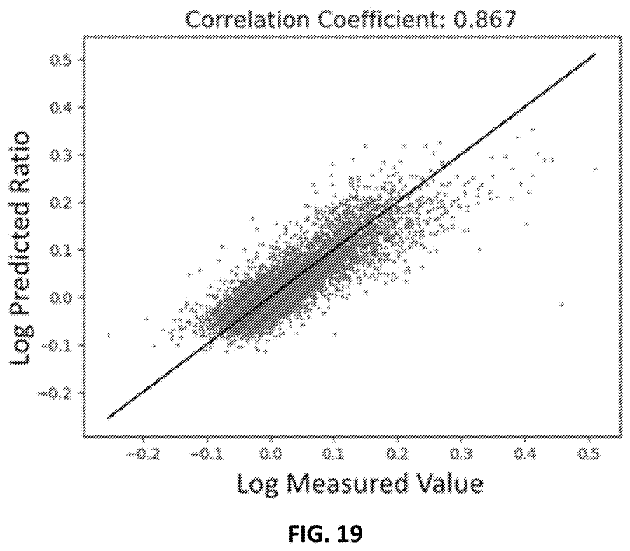

FIG. 19 shows an example of a prediction of the z-score between diaphorase with and without FAD bound, and demonstrates that the subtle effect on the molecular recognition pattern of binding a cofactor can be represented quantitatively using the same approach. Here, instead of using the binding value itself in the training, for each peptide in the array, a z-score between diaphorase with and without FAD bound was calculated (the ratio of the difference in mean between sample sets divided by the square root of the sum of the squares of the standard deviation). While the fit is not as good, it is still close to the error in the measurement itself (the error is larger here because we are looking at relatively small differences between larger numbers). This is a potential pathway to finding a peptide that would either interfere with the binding of a normal ligand for a protein or would stabilize the binding.

Turning to FIG. 8, an example 800 of hardware for predicting functions of molecular libraries that can be used in accordance with some embodiments of the disclosed subject matter is shown. As illustrated, hardware 800 can include one or more server(s) 802, a communication network 804, and a user device 806.

Server(s) 802 can be any suitable server(s) for predicting functions of molecular sequences. For example, in some embodiments, server(s) 802 can store any suitable information used to train a neural network to predict functions of molecular sequences. As a more particular example, in some embodiments, server(s) 802 can store sequence information (e.g., amino acid sequences of peptides, and/or any other suitable sequence information). As another more particular example, in some embodiments, server(s) 802 can store data and/or programs used to implement a neural network. In some embodiments, server(s) 802 can implement any of the techniques described above in connection with FIGS. 1-7 and 9-20. In some embodiments, server(s) 802 can be omitted.

Communication network 804 can be any suitable combination of one or more wired and/or wireless networks in some embodiments. For example, communication network 804 can include any one or more of the Internet, a mobile data network, a satellite network, a local area network, a wide area network, a telephone network, a cable television network, a WiFi network, a WiMax network, and/or any other suitable communication network.

In some embodiments, user device 806 can include one or more computing devices suitable for predicting functions of molecular sequences, and/or performing any other suitable functions. For example, in some embodiments, user device 806 can store any suitable data or information for implementing and/or using a neural network to predict functions of molecular sequences. As a more particular example, in some embodiments, user device 806 can store and/or use sequence information (e.g., sequences of amino acids in peptides, and/or any other suitable information), data and/or programs for implementing a neural network, and/or any other suitable information. In some embodiments, user device 806 can implement any of the techniques described above in connection with FIGS. 1-7 and 9-20. In some embodiments, user device 806 can be implemented as a laptop computer, a desktop computer, a tablet computer, and/or any other suitable type of user device.

Although only one each of server(s) 802 and user device 806 are shown in FIG. 8 to avoid over-complicating the figure, any suitable one or more of each device can be used in some embodiments.

Server(s) 802 and/or user device 806 can be implemented using any suitable hardware in some embodiments. For example, in some embodiments, devices 802 and 806 can be implemented using any suitable general purpose computer or special purpose computer. For example, a server may be implemented using a special purpose computer. Any such general purpose computer or special purpose computer can include any suitable hardware. For example, as illustrated in example hardware 900 of FIG. 9, such hardware can include hardware processor 902, memory and/or storage 904, an input device controller 906, an input device 908, display/audio drivers 910, display and audio output circuitry 912, communication interface(s) 914, an antenna 916, and a bus 918.

Hardware processor 902 can include any suitable hardware processor, such as a microprocessor, a micro-controller, digital signal processor(s), dedicated logic, and/or any other suitable circuitry for controlling the functioning of a general purpose computer or a special purpose computer in some embodiments.

Memory and/or storage 904 can be any suitable memory and/or storage for storing programs, data, and/or any other suitable information in some embodiments. For example, memory and/or storage 904 can include random access memory, read-only memory, flash memory, hard disk storage, optical media, and/or any other suitable memory.

Input device controller 906 can be any suitable circuitry for controlling and receiving input from a device in some embodiments. For example, input device controller 906 can be circuitry for receiving input from a touch screen, from one or more buttons, from a voice recognition circuit, from a microphone, from a camera, from an optical sensor, from an accelerometer, from a temperature sensor, from a near field sensor, and/or any other type of input device.

Display/audio drivers 910 can be any suitable circuitry for controlling and driving output to one or more display/audio output circuitries 912 in some embodiments. For example, display/audio drivers 910 can be circuitry for driving an LCD display, a speaker, an LED, or any other type of output device.

Communication interface(s) 914 can be any suitable circuitry for interfacing with one or more communication networks, such as network 804 as shown in FIG. 8. For example, interface(s) 914 can include network interface card circuitry, wireless communication circuitry, and/or any other suitable type of communication network circuitry.

Antenna 916 can be any suitable one or more antennas for wirelessly communicating with a communication network in some embodiments. In some embodiments, antenna 916 can be omitted when not needed.

Bus 918 can be any suitable mechanism for communicating between two or more components 902, 904, 906, 910, and 914 in some embodiments.

Any other suitable components can be included in hardware 900 in accordance with some embodiments.

It should be understood that at least some of the above described blocks of the processes of FIGS. 1-4 and 20 can be executed or performed in any order or sequence not limited to the order and sequence shown in and described in the figures. Also, some of the above blocks of the processes of FIGS. 1-4 and 20 can be executed or performed substantially simultaneously where appropriate or in parallel to reduce latency and processing times. Additionally or alternatively, some of the above described blocks of the processes of FIGS. 1-4 and 20 can be omitted.

In some embodiments, any suitable computer readable media can be used for storing instructions for performing the functions and/or processes herein. For example, in some embodiments, computer readable media can be transitory or non-transitory. For example, non-transitory computer readable media can include media such as non-transitory magnetic media (such as hard disks, floppy disks, and/or any other suitable magnetic media), non-transitory optical media (such as compact discs, digital video discs, Blu-ray discs, and/or any other suitable optical media), non-transitory semiconductor media (such as flash memory, electrically programmable read-only memory (EPROM), electrically erasable programmable read-only memory (EEPROM), and/or any other suitable semiconductor media), any suitable media that is not fleeting or devoid of any semblance of permanence during transmission, and/or any suitable tangible media. As another example, transitory computer readable media can include signals on networks, in wires, conductors, optical fibers, circuits, any suitable media that is fleeting and devoid of any semblance of permanence during transmission, and/or any suitable intangible media.

Accordingly, methods, systems, and media for predicting functions of molecular sequences are provided.

Although the invention has been described and illustrated in the foregoing illustrative embodiments, it is understood that the present disclosure has been made only by way of example, and that numerous changes in the details of implementation of the invention can be made without departing from the spirit and scope of the invention, which is limited only by the claims that follow. Features of the disclosed embodiments can be combined and rearranged in various ways.

1. A method for predicting functions of molecular sequences, comprising:
generating an array that represents a sequence of molecules;
determining a projection of the sequence of molecules, wherein the determining comprises multiplying a representation of the array that represents the sequence of the molecules by a first hidden layer matrix that represents a number of possible sequence dependent functions, wherein the first hidden layer matrix is determined during training of a neural network; and
determining a function of the sequence of molecules by applying a plurality of weights to a representation of the projection of the sequence of molecules, wherein the plurality of weights is determined during the training of the neural network.

2. The method of claim 1, wherein the sequence of molecules is a sequence of peptides.

3. The method of claim 2, wherein the representation of the array that represents the sequence of molecules indicates amino acids of peptides in the sequence of peptides.

4. The method of claim 3, wherein the representation of the array that represents the sequence of molecules includes binary values that each indicate whether an amino acid of a plurality of amino acids is present in a peptide in the sequence of peptides.

5. The method of claim 4, wherein the binary representation of the sequence is converted to a real value representation via multiplication by an intermediate matrix.

6. The method of claim 1, wherein the function of the sequence of the molecules is a binding value of a protein to each molecule in the sequence of molecules.

7. The method of claim 1, wherein the determining the projection of the sequence of molecules further comprises multiplying the product of the representation of the array that represents the sequence of the molecules and the first hidden layer matrix that represents a number of possible sequence dependent functions by a second hidden layer matrix.

8. The method of claim 1, further comprising applying an activation function to the projection of the sequence of molecules to generate the representation of the projection of the sequence of molecules.

9. The method of claim 1, wherein the sequence of molecules is a sequence of nucleotides, peptide nucleic acid monomers, or peptoid monomers

10. The method of claim 1, wherein the first hidden layer matrix is an eigensequence matrix.

11. A system for predicting functions of molecular sequences, comprising:
a memory; and
a hardware processor coupled to the memory and configured to:
generate an array that represents a sequence of molecules;
determine a projection of the sequence of molecules, wherein the determining comprises multiplying a representation of the array that represents the sequence of the molecules by a first hidden layer matrix that represents a number of possible sequence dependent functions, wherein the first hidden layer matrix is determined during training of a neural network; and
determine a function of the sequence of molecules by applying a plurality of weights to a representation of the projection of the sequence of molecules, wherein the plurality of weights is determined during the training of the neural network.

12. The system of claim 11, wherein the sequence of molecules is a sequence of peptides.

13. The system of claim 12, wherein the representation of the array that represents the sequence of molecules indicates amino acids of peptides in the sequence of peptides.

14. The system of claim 13, wherein the representation of the array that represents the sequence of molecules includes binary values that each indicate whether an amino acid of a plurality of amino acids is present in a peptide in the sequence of peptides.

15. The system of claim 14, wherein the binary representation of the sequence is converted to a real value representation via multiplication by an intermediate matrix.

16. The system of claim 11, wherein the function of the sequence of the molecules is a binding value of a protein to each molecule in the sequence of molecules.

17. The system of claim 11, wherein the determining the projection of the sequence of molecules further comprises multiplying the product of the representation of the array that represents the sequence of the molecules and the first hidden layer matrix that represents a number of possible sequence dependent functions by a second hidden layer matrix.

18. The system of claim 11, wherein the hardware processor is further configured to apply an activation function to the projection of the sequence of molecules to generate the representation of the projection of the sequence of molecules.

19. The system of claim 11, wherein the sequence of molecules is a sequence of nucleotides, peptide nucleic acid monomers, or peptoid monomers.

20. The system of claim 11, wherein the first hidden layer matrix is an eigensequence matrix.
